(19) United States
(12) Patent Application Publication
(10) Pub. No.: US 2010/0330588 A1
(43) Pub. Date: Dec. 30, 2010

(54) METHOD TO QUANTIFY METHYLTRANSFERASE ACTIVITY

(75) Inventor: Joan Hevel, Logan, UT (US)

Correspondence Address: UTAH STATE UNIVERSITY;TECHNOLOGY COMMERCIALIZATION OFFICE, 570 RESEARCH PARK WAY, SUITE 101, NORTH LOGAN, UT, 84341, US

(73) Assignee: Utah State University, North Logan, UT (US)

(21) Appl. No.: 12/825,548

(22) Filed: Jun. 29, 2010

Related U.S. Application Data
(60) Provisional application No. 61/221,453, filed on Jun. 29, 2009.

(51) Int. Cl. G01N 33/573

(52) U.S. Cl. 435/7.4

(57) ABSTRACT

The related disclosure pertains to a method to quantify methyltransferase activity. In an exemplar embodiment, the method may be carried out by the following steps: i) incubating a mixture comprising a methyltransferase, a substrate, and radiolabeled S-adenosylmethionine for a period of time sufficient for the protein methyltransferase to methylate and transform at least some of the substrate into a methylated product of the substrate, then ii) at one or more times removing an aliquot of the mixture, then iii) contacting the aliquot of the mixture to a resin capable of binding the methylated product of the substrate, followed by iv) a washing step to remove the unreacted radiolabeled S-adenosylmethionine, followed by v) an elution step to elute and isolate the methylated product, then vi) measuring the amount of radiolabel in the methylated product.

### CROSS-REFERENCE TO RELATED APPLICATIONS

This application claims the benefit of U.S. Provisional Application No. 61221453, entitled “Method to Quantify Methyltransferase Activity,” filed on Jun. 29, 2009.

### STATEMENT REGARDING FEDERALLY SPONSORED RESEARCH OR DEVELOPMENT

Not Applicable

### REFERENCE TO SEQUENCE LISTING, A TABLE, OR A COMPUTER PROGRAM LISTING COMPACT DISK APPENDIX

Not Applicable

### DEFINITIONS

The following terms and phrases are defined as follows:
“Methyltransferase activity,” as used herein, means the transferring of one or more methyl groups from a donor molecule to an acceptor molecule.
“Methyltransferase,” as used herein, means an enzyme that participates in the transferring of one or more methyl groups from a donor molecule to an acceptor molecule.
“Protein methyltransferase,” as used herein, means an enzyme capable of transferring a methyl group from a donor to an acceptor, where the acceptor is a protein. In other words, a protein methyltransferase is a methyltransferase capable of methylating a protein.
“Substrate,” as used herein, means a molecule upon which an enzyme acts; for example, the molecule that is methylated by a methyltransferase enzyme.
“Product,” as used herein, means a chemical substance formed as a result of a chemical reaction; for example, the product produced by the action of a methyltransferase is the methylated form of the substrate.
“The methylated product of the substrate,” as used herein, specifically refers to a second molecule, derived from a first molecule, where the essential difference between the second and first molecule comprises the presence of one or more methyl groups on the second molecule, and where the presence of the one or more methyl groups is the result of methyltransferase activity.
“S-adenosylmethionine,” as used herein, means (S,S)-adenosyl-L-methionine and (R,S)-adenosyl-L-methionine. Equivalent names, terms, phrases, or abbreviations include “SAM” and “Adomet.”
“Radiolabeled S-adenosyl-L-methionine,” as used herein, means any radioactive form of S-adenosyl-L-methionine.
“S-adenosylhomocysteine,” is a byproduct of S-adenosylmethionine dependent methyltransferase activity. Equivalent names, terms, phrases, or abbreviations include “AdoHcy.”
“Aliquot,” as used herein, means the portion of a solution that is to be used in a process or method, and can refer to either a portion of the total amount of a solution or the whole amount of a solution.
“Contacting,” as used herein, means to bring to the state or condition of touching or of being in immediate proximity. For example, litmus paper turns red on contact with an acid.
“Resin,” as used herein, means a separation media or medium. Without limiting the use of the term, common resins include reverse phase resins, chromatography resins, and gel filtration resins.
“Binding,” as used herein, means the reversible association of molecules in pharmacology, biochemistry, or chemistry; for example, the binding of a ligand to a receptor, or the binding of a metal ion by a chelator.
“Methylated,” as used herein, means having received one or more methyl groups. Without limiting the term in any way, it is meant to specifically include the replacement of a hydrogen atom with a methyl group and to specifically refer to proteins, peptides, small molecules and nucleic acids that have received a methyl group.
“Washing,” as used herein, means to separate the desired products of a chemical or biological reaction or process, from the undesired products, byproducts, or unreacted reagents and components.
“Scintillation cocktail,” as used herein, means a solution of organic solvent along with a fluor which fluoresces when struck by ionizing radiation, and can then be detected by a liquid scintillation counter.
“Liquid Scintillation Counter,” as used herein, means a laboratory machine for measuring ionizing radiation. Commonly, the sensor, called a scintillator, consists of a transparent crystal, usually phosphor, plastic (usually containing anthracene), or organic liquid (see liquid scintillation counting) that fluoresces when struck by ionizing radiation; a sensitive photomultiplier tube (PMT) measures the light from the crystal; the PMT may be attached to an electronic amplifier and other electronic equipment to count and possibly quantify the amplitude of the signals produced by the photomultiplier. Equivalent names, terms, phrases, or abbreviations include “LSC.”
“Scintillation Counting,” as used herein, refers to a standard laboratory method for measuring radiation from beta-emitting nuclides.
“Nuclides,” as used herein, means is an atom with an unstable nucleus, which is a nucleus characterized by excess energy which is available to be imparted either to a newly-created radiation particle within the nucleus, or else to an atomic electron.
“Spotting,” as used herein, means the placing of a solution, an aliquot of a solution, or a drop of a solution onto a membrane, piece of paper, or other permeable sheet, film, or strip thereof.
“Membrane,” as used herein, means a thin sheet of natural or synthetic material that is permeable to substances in solution
“Proximity assay,” as used herein, means a Scintillation Proximity Assay or SPA, which eliminates the separation step of classic Radioimmunoassay (RIA) and is performed using low energy radioisotopes (³H and ¹²⁵I) as labels due to their short range electron emission. When bound close to a solid scintillator surface during the binding reaction, electron energy is transferred to the scintillator and the resulting photons can be captured by scintillation counting. Electrons emitted from labeled molecules not close to the surface dissipate their energy and are not detected
“Pipette,” as used herein, means a laboratory instrument used to transport a measured volume of liquid. Equivalent names, terms, phrases, or abbreviations include “pipet”, “pipettor” or “chemical dropper.”
“Pipette tip,” as used herein, means an end piece capable of attaching to a pipette, into which a liquid solution can be drawn and from which the liquid solution can be subsequently expelled.
“PRMT,” as used herein, means a protein arginine methyltransferase.
“Autoradiography,” as used herein, means a technique in which radioactive molecules make their location known by exposing photographic films or emulsions.
“S-adenosyl-L-[methyl-³H]methionine,” as used herein, means a tritium radiolabeled S-adenosyl-L-methionine.
“S-adenosyl-L-[methyl-¹⁴C]methionine,” as used herein, means carbon 14 radiolabeled S-adenosyl-L-methionine.
“incorporated ³H-methyl,” as used herein, means one or more tritium labeled methyl groups present in the protein product of methyltransferase activity. It is equivalent to “incorporated tritium.”
“ZipTip,” as used herein, refers to a commercially available resin packed pipette tip useful in practicing the present invention. The use of the term “ZipTip” is not meant to limit the use of the inventive concept to a particular product or type of resin.
“[³H]-AdoMet,” as used herein, means S-adenosyl-L-[methyl-³H]methionine.
“AdoMet,” as used herein, means S-adenosyl-L-methionine.

### BACKGROUND OF THE INVENTION

The present invention is in the technical field of molecular biology. More particularly, the present invention is in the technical field of molecular biology techniques.

Methyltransferases are a widespread class of enzymes present in both prokaryotes and eukaryotes. Methyltransferases function by transferring one or more methyl groups from a donor molecule to an acceptor molecule. One of the most common protein methylations takes place on the nitrogens of lysine, histidine, asparagine, glutamine, and arginine. Methylation of the terminal guanidino nitrogens of arginine occurs via the protein arginine methyltransferases (PRMTs), a family of eleven human enzymes that target both nuclear and cytosolic proteins.

Within the art there are generally two approaches to measuring methyltransferase activity. The first is to measure the by-product of S-adenosyl-L-methionine dependent methyltransferase activity, S-adenosylhomocysteine (AdoHcy). The second is to directly measure the methylated product of S-adenosyl-L-methionine dependent methyltransferase activity. Several novel assays have been developed in recent years to measure AdoHcy formation. The current methods for measuring AdoHcy formation are commonly limited to using peptide substrates, and may not be of use when the substrates to be methylated are proteins. Moreover, such methods are not compatible when the investigator wishes to determine the inhibitory effect of AdoHcy on enzyme activity.

One of the most common methods for analyzing methylated proteins involves the use of radiolabeled S-adenosyl-L-methionine. Commonly used forms of radiolabeled S-adenosyl-L-methionine include S-adenosyl-L-[methyl-³H]methionine and S-adenosyl-L-[methyl-¹⁴C]methionine. This method requires the separation of unreacted radiolabeled S-adenosyl-L-methionine from radiolabeled protein products, followed by detection of radiolabeled proteins. SDS-PAGE is commonly used to separate unreacted radiolabeled S-adenosyl-L-methionine from radiolabeled protein products. Although the aforementioned separation step is usually easily achieved, the detection of the radiolabeled proteins is known to be time consuming, especially when the specific activity of the radiolabeled S-adenosyl-L-methionine is low or few residues of the protein have been methylated.

Two methods commonly used for the detection of the radiolabeled proteins are summarized in scheme 1.

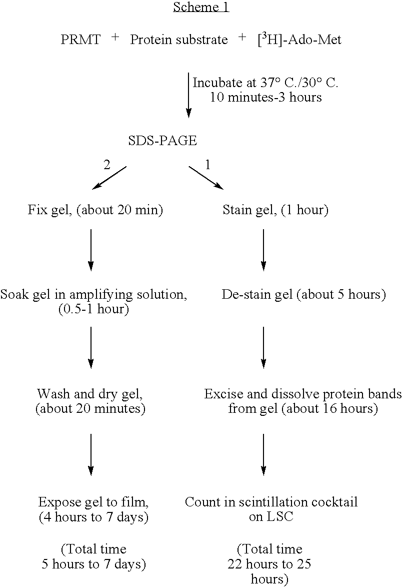

In the method 1 of scheme 1, protein bands in acrylamide are crushed, the protein is extracted, and radiolabel associated with the proteins is detected by liquid scintillation counting. This method is known to be laborious and tedious, taking on the order of greater than 12 hours to obtain efficient retrieval of the proteins from the acrylamide. In the method 2 of scheme 1, radiolabeled proteins are detected, either in-gel or post-transfer to a membrane, by phosphorimaging/fluorography. Depending on the number of protein residues methylated and the specific activity of the radiolabeled S-adenosyl-L-methionine, the detection by phosphorimaging/fluorography commonly takes several weeks to months. A standard curve that relates band intensity to moles of radiolabeled methyl groups must also be done in order to provide quantitative results.

Methods that are used to directly detect methylated protein products include protein hydrolysis and amino acid analysis of released N-methyl amino acids, detection by specific antibodies, and mass spectroscopy. These methods are either labor intensive, limited to commercially available antibodies, or require specialized equipment needing technical expertise.

Current methods to assay for methyltransferase activity have several drawbacks, including the production of large amounts of radioactive waste, high cost, time consuming methods, and limited sensitivity. Additionally, there is no easy method for directly measuring the methylated product. These limitations have long been a problem for researchers interested in studying methyltransferase activity. There is a long-standing need in the art for a simple, quick, easy method to assay for methyltransferase activity.

### BRIEF SUMMARY OF THE INVENTION

The present invention relates to novel methods useful for conducting methylation assays. One embodiment of the present invention is shown in scheme 2.

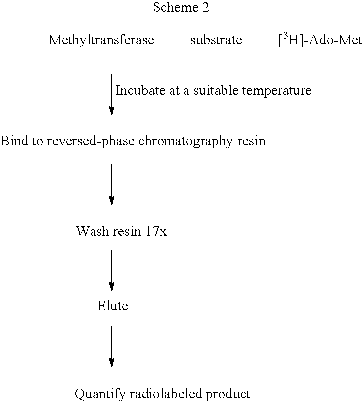

Optionally, the time-consuming step of separating unreacted radiolabeled S-adenosylmethionine from radiolabeled protein products has been alleviated (see, for example, scheme 1 and scheme 2). Optionally, unlabeled S-adenosylmethionine can be added to reactions to furnish saturating concentrations of S-adenosylmethionine while maintaining sufficient detection sensitivity, even at short reaction times. In one embodiment, the use of nanomolar enzyme concentrations and low protein substrate concentrations provides for a method highly suitable for measuring initial rates. Optionally, the novel methods described herein provide for reduction in the amount of radioactive waste generated. Compared to prior art methods, the reduction in the generation of radioactive waste may be up to a 3000-fold reduction. Without limiting the invention, utility of the novel methods described herein is demonstrated by the rapid generation of a Michaelis-Menten curve for the methylation of hnRNP K protein by hPRMT 1v1. Again, without limiting the invention, further utility is demonstrated by the evaluating of the effect of a GST (glutathione S-transferase) tag on the activity of PRMT6.

Scheme 3 is an embodiment of the disclosed method, which uses a protein methyltransferase and a protein substrate.

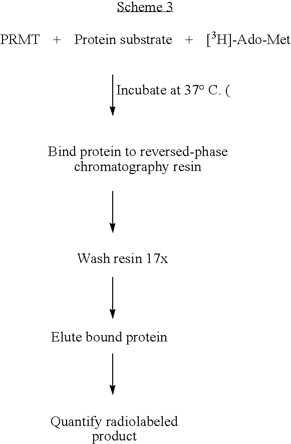

Optionally, the novel methods described herein provide for, i) separation of unreacted S-adenosylmethionine and methylated protein that is extremely fast, and, ii) direct measurement of protein methylation using liquid scintillation counting, which may increase the ability to detect small amounts of radiolabel and to conduct measurements of steady state kinetics, and, iii) in certain embodiments, a protein methylation assay with a lower limit of detection of about 0.12 nM CH₃/minute.

### BRIEF DESCRIPTION OF THE SEVERAL VIEWS OF THE DRAWING

FIG. 1 shows elution profiles of radioactivity showing the separation of unreacted radiolabeled S-adenosylmethionine from incorporated ³H-methyl using reverse phase chromatography resin.

FIG. 2 shows the binding and elution efficiencies of the reverse phase chromatograph resin packed pipet tips.

FIG. 3 shows quantitative measurement of the rate of hPRMT1v1-catalyzed methylation of hnRNP K protein using the reverse phase chromatography resin methods described herein.

FIG. 4 shows a comparison of the methylation activities of GST-PRMT6 and His-PRMT6 using the reverse phase chromatography resin methods described herein.

### DETAILED DESCRIPTION OF THE INVENTION

In broad embodiment, the present invention is a method to measure methyltransferase activity, including the presence or absence of methyltransferase activity, or the enhancement or inhibition of methyltransferase activity, by using a resin to bind or separate the protein product of methyltransferase activity from other reactants or components of a methyltransferase reaction mixture. In one embodiment, the invention can be practiced as shown in Scheme 2, as follows:

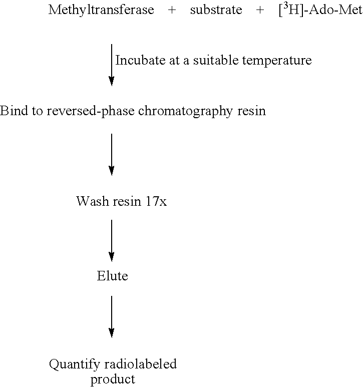

Generally, the method may be carried out by the following steps: i) incubating a mixture comprising a methyltransferase, a substrate, and radiolabeled S-adenosylmethionine for a period of time sufficient for the protein methyltransferase to methylate and transform at least some of the substrate into a methylated product of the substrate, then ii) at one or more times removing an aliquot of the mixture, then iii) contacting the aliquot of the mixture to a resin capable of binding the methylated product of the substrate, followed by iv) a washing step to remove the unreacted radiolabeled S-adenosylmethionine, followed by v) an elution step to elute and isolate the methylated product, then vi) measuring the amount of radiolabel in the methylated product. Alternatively, the following steps may be used: i) incubating a mixture comprising a methyltransferase, a substrate, and radiolabeled S-adenosylmethionine for a period of time sufficient for the protein methyltransferase to methylate and transform at least some of the substrate into a methylated product of the substrate, then ii) at one or more times removing an aliquot of the mixture, then iii) contacting the aliquot of the mixture to a resin capable of binding the methylated product of the substrate, followed by iv) a washing step to remove the unreacted radiolabeled S-adenosylmethionine, followed by v) measuring the amount of radiolabel in the methylated product without eluting the methylated product from the resin, such that measurement of radiolabel in methylated product occurs with the methylated product still bound to the resin. Optionally, the above described methods can be followed with a measuring of the amount of radiolabel and correlating the amount of radiolabel in the methylated product to the amount of the methylated product.

Resins that may be used in the present invention include, but are not limited to, reverse phase resin, ion exchange resin, and gel filtration resins. Without limiting the invention, resins may be contained, for example, in pipette tips or multi-well plates.

Measuring the amount of radiolabel associated with the radiolabeled substrate may be done by scintillation counting on a LSC. Alternatively, the measuring the amount of radiolabel in the methylated product is done by spotting the methylated product of the substrate on a membrane and developing the membrane by autoradiography. In yet another alternative, the measuring the amount of radiolabel associated with the radiolabeled substrate is done by proximity assay.

In certain embodiments, the resin used is contained in a pipette tip, and, the methyltransferase is a PRMT, and, optionally, capable of methylating an amino acid from a group comprising lysine, histidine, asparagines, glutamine, and arginine, and, the incubating a mixture contains a protein methyltransferase, a substrate and radiolabeled S-adenosylmethionine and incubation is done for a period of time between 0 and 12 minutes, and, the contacting of the aliquot of the mixture to a resin capable of binding to methylated substrate is done for a period of time less than 2 minutes, and, the substrate is a member of a group including, but not limited to, small molecules, peptides, proteins, and nucleic acids, and, the S-adenosylmethionine is S-adenosyl-L-methionine.

In certain embodiments, the S-adenosylmethionine used is S-adenosyl-L-methionine, the methyltransferase is one specific for a desired substrate and the isolation and elution of the protein product of methyltransferase activity is carried out using a pipette tip packed with reverse phase chromatography resin. In an alternative embodiment a multi-well plate lined or packed with reverse phase chromatography resin is used to isolate, separate, or purify the protein product of methyltransferase activity. The latter embodiment may be especially useful in the screening of compounds for their effects on methyltransferase activity and is also compatible with other types of resin disclosed herein.

An embodiment of the disclosed method using a protein methyltransferase and a protein substrate is shown is Scheme 3, as follows:

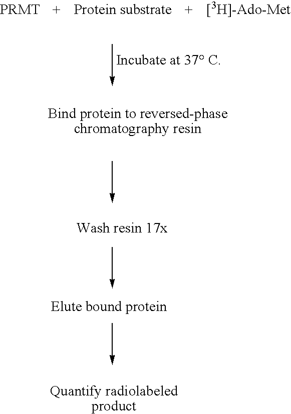

The embodiment of scheme 3 may be altered and practiced within the scope of any of the broader steps of scheme 2.

The following materials and methods can be used to practice the disclosed invention:

### Materials and Methods

### Materials

Commercially available pipette tips with reversed-phase (C4) chromatography resin fixed at the tip were used. For example, without limiting the invention, the pipette tips used included the ZipTipC₄, pipette tip (Millipore). Tagless human PRMT1 variant 1 (hPRMT1v1) was expressed and purified. Histone H3.3 was purchased from New England Biolabs. His-hnRNP K was expressed and purified. S-adenosyl-L-[methyl-³H]methionine (specific activity 67.3 Ci/mmol, 0.55 μCi/μl) was purchased from Perkin Elmer.

### Expression and Purification of His-PRMT6

E. coli BL21 (Codon plus) cells overexpressing His-PRMT6 were grown in 1 L Luria-Bertani (LB) broth at 37° C. to an OD₆₀₀ of 0.6. Protein expression was induced with 1 mM isopropy-β-D-thiogalactopyranoside at 22° C. for 24 hours. Cells were harvested by centrifugation and resuspended in 30 ml loading buffer (50 mM sodium phosphate, 300 mM NaCl and 10 mM Imidazole; pH 7.4). Cells were lysed by sonication and cell debris was removed by centrifugation at 12,000 g at 4° C. for 10 min. The supernatant was incubated with 3 ml packed HisPur cobalt resin (Pierce) at 4° C. with end-over-rotation for 3 hours. The resin was washed with ˜300 ml of loading buffer. Bound His-PRMT6 was eluted using a linear imidazole gradient from 10-150 mM imidazole in loading buffer. Fractions showing the presence of His-PRMT6 by SDS-PAGE analysis were concentrated using an Amicon Ultra centrifugal filter device with a 30 kDa molecular weight cutoff. Buffer was exchanged to buffer containing 50 mM sodium phosphate, pH 8.0 according to the manufacturer's instructions. His-PRMT 6 was further purified using a MonoQ column (GE healthcare). His-PRMT6 was loaded on to the column in 50 mM sodium phosphate, pH 8.0. The column was washed with loading buffer until the absorbance at 280 nm approached the baseline. Bound protein was then eluted using a stepwise salt gradient from 0 to 1.0 M NaCl. Fractions were analyzed by SDS and fractions containing His-PRMT6 were pooled and concentrated as above and buffer exchanged to loading buffer. Protein was stored at −80° C. in 10% glycerol. Approximately 70 μg of purified protein was obtained from 1 L of LB broth.

### Expression and Purification of GST-PRMT6

E. coli BL21 (Codon plus) cells overexpressing GST-PRMT6 were grown in 1 L LB broth at 37° C. to an OD₆₀₀ of 0.6. Protein expression was induced with 1 mM isopropyl-β-D-thiogalactopyranoside at 22° C. for 24 hours. Cells were harvested by centrifugation and resuspended in 60 ml ice-cold phosphate buffered saline (PBS). Cells were pelleted by centrifugation at 3000 g at 4° C. for 10 min, the supernatant was discarded and cells resuspended in 60 ml ice-cold PBS. Cells were lysed by sonication and cell debris was removed by centrifugation at 12,000 g at 4° C. for 10 min. The supernatant was incubated with 4 ml of GST resin slurry (GenScript) at 4° C. with end-over-rotation for 3 hours. The resin was washed with ˜200 ml of PBS and bound GST-PRMT6 eluted using a linear gradient from 0-10 mM reduced glutathione in 50 mM Tris-HCL, pH 8.0. Fractions containing GST-PRMT6 were concentrated using an Amicon Ultra centrifugal filter device with a 30 kDa molecular weight cutoff. Buffer was exchanged to buffer containing 50 mM sodium phosphate, pH 8.0 according to the manufacturer's instructions. GST-PRMT 6 was further purified using a MonoQ column (GE healthcare). GST-PRMT6 was loaded on to the column in 50 mM sodium phosphate, pH 8.0. The column was washed with loading buffer until the absorbance at 280 nm approached the baseline. Bound protein was then eluted using a stepwise salt gradient from 0 to 1.0 M NaCl. Fractions were treated as described above. Protein was stored at −80° C. in 10% glycerol. Approximately 100 μg of purified protein was obtained from 1 L of LB broth.

### Composition of the Methyltransferase Reaction for the Determination of PRMT Activity

Assays were performed at 37° C., in a final volume of 125 μl. Each assay contained 1.0 μM unlabeled S-adenosyl-L-methionine (AdoMet), 0.6 μCi/μl S-adenosyl-L-[methyl-³H]methionine (specific activity 67.3 Ci/mmol, 0.55 μCi/μl, Perkin Elmer), 100 mM Hepes, 0.38 μM bovine serum albumin (BSA), 10% glycerol, 10 nM AdoHcy Nucleosidase (MTAN), and 1.7 μM protein substrate; pH of reaction was 7.6. Each reaction was equilibrated at 37° C. for 3 min and then initiated with 100 nM PRMT. Samples (10 μL) were removed at specified times and were analyzed using either SDS-PAGE or methods related to the present disclosure, as discussed below.

### Separation of [³H]-AdoMet and Methylated Proteins by SDS-PAGE

A 10 L aliquot of the reaction mixture was transferred to 5 μl 4×SDS sample buffer to quench the reaction. Unreacted [³H]-AdoMet was separated from the methylated protein products by SDS-PAGE. Protein was transferred to a PVDF membrane and methylation was detected in one of two ways. The membrane was sprayed with EN³HANCE (Perkin Elmer) according to the manufacturer's instructions, and methylation was detected by fluorography after incubating with film for 10 weeks. Multi Gauge software V2.3 (FujiFilms) was used to quantitate band intensity.

### Separation of [³H]-AdoMet and Methylated Proteins by a Novel Method Using Reverse Phase Chromatography Resin.

A 10 L aliquot of the reaction mixture was transferred to 6 μl of sample preparation buffer (8 M Guanidine-HCL in 2.5% trifluoroacetic acid (TFA)), making the final concentrations 3 M Guanidine-HCL and 0.94% TFA in the mixture. Guanidine-HCl denatures the protein and TFA makes the reaction very acidic so that PRMT activity is quenched. A pipette tip containing reversed-phase (C4) chromatography resin fixed at the tip, one for each time point, was then pre-wetted in wetting buffer (75% acetonitrile in deionized H₂O) by pipetting up and down twice. The pipette tip was equilibrated by washing twice with equilibration buffer (0.1% TFA in deionized H₂O). Using the pre-equilibrated pipette tip, each quenched reaction was pipetted up and down 20 times, so as to bind the protein in the reaction to the C4 resin. The pipette tip was washed 17 times with 10 μl wash buffer (0.1% TFA in deionized water) to remove unreacted [³H]-AdoMet. Bound protein was eluted by aspirating and dispensing 10 μl of elution buffer (75% acetonitrile in 0.1% TFA) 10 times in the same tube. Eluate was dispensed into 20 ml of scintillation cocktail (Fisher Scientific) and counted in a LSC.

The forgoing materials and methods are useful in practicing certain embodiments of the disclosed invention, but are not presented as limiting the invention. Once presented with the current disclosure, one skilled in the art may readily recognize modifications, adaptations, or changes to the methods or components useful in the methods described herein, and it is the disclosure of certain embodiments and options, combined with what one skilled in the art would readily add to embodiments and options, which comprise the present invention.

Referring once again to scheme 1, scheme 2, and scheme 3, there is shown a comparison of currently used methods (scheme 1) for measuring methylation to the reverse phase chromatography resin method disclosed herein (scheme 2 and 3). Referring now to scheme 1, total time for analysis by method 1 of scheme 1 reflects conditions used to monitor a single end-point, usually under conditions of long enzyme incubation times. Short reaction times required to collect initial rate data necessitate much longer exposure times. Referring now to scheme 2, there is shown a general method for an improved methyltransferase assay. Referring to scheme 3, there is shown a novel method to quantify protein methyltransferase activity. Still referring to scheme 3, the separation of unreacted S-adenosyl-L-methionine and methylated protein product is extremely fast when using the reverse phase chromatography resin.

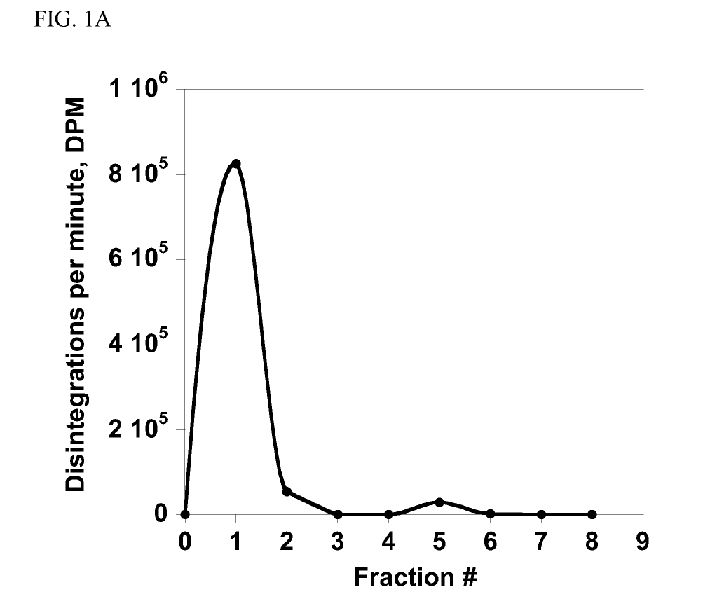

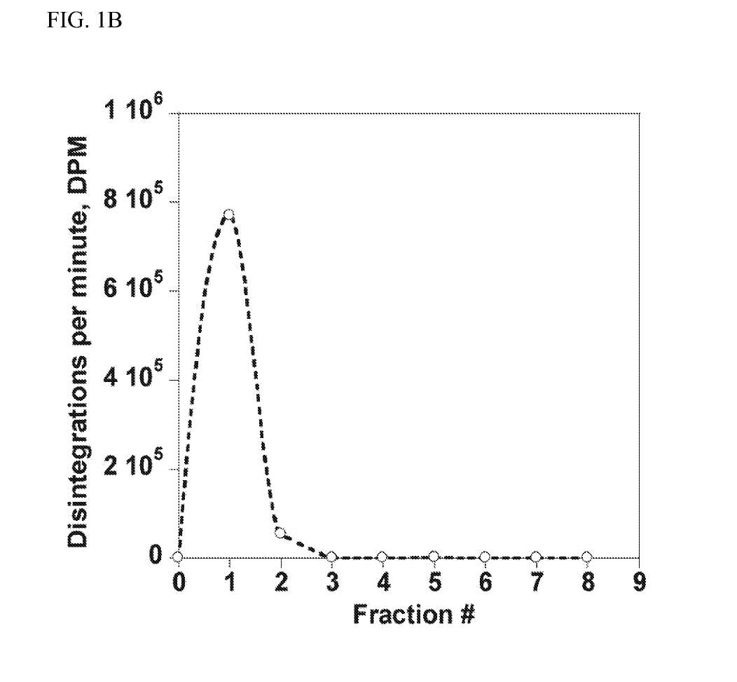

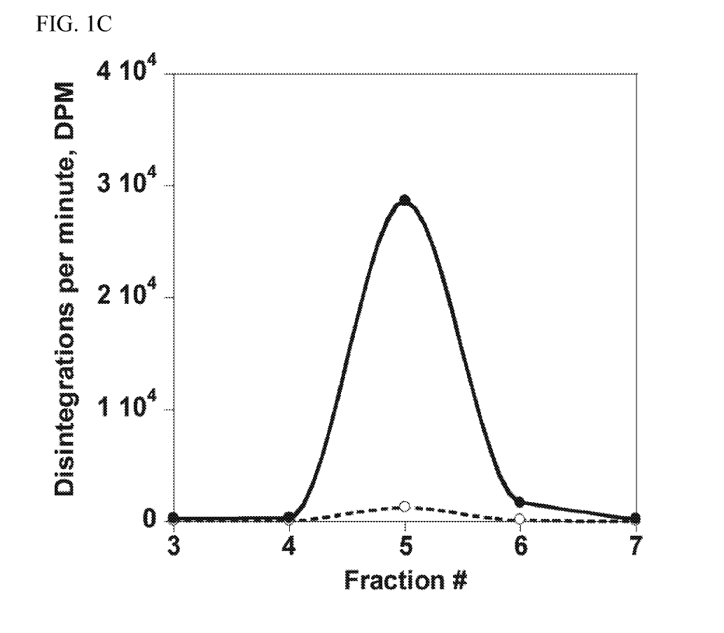

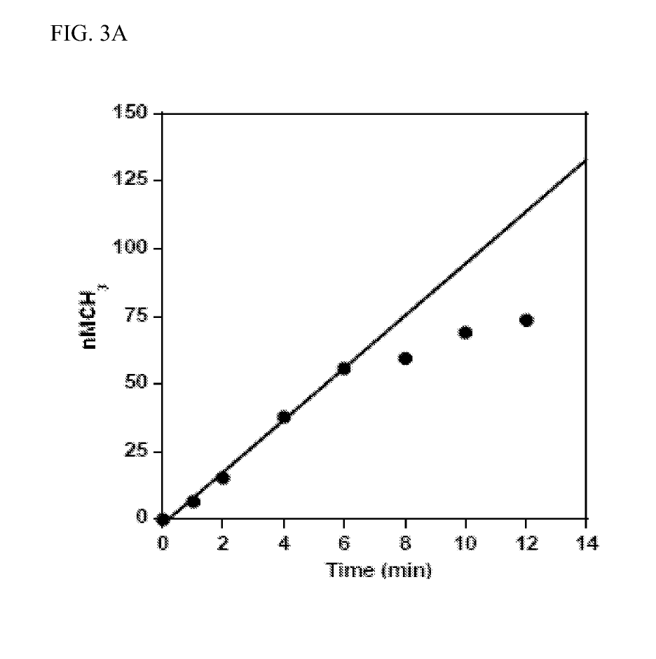

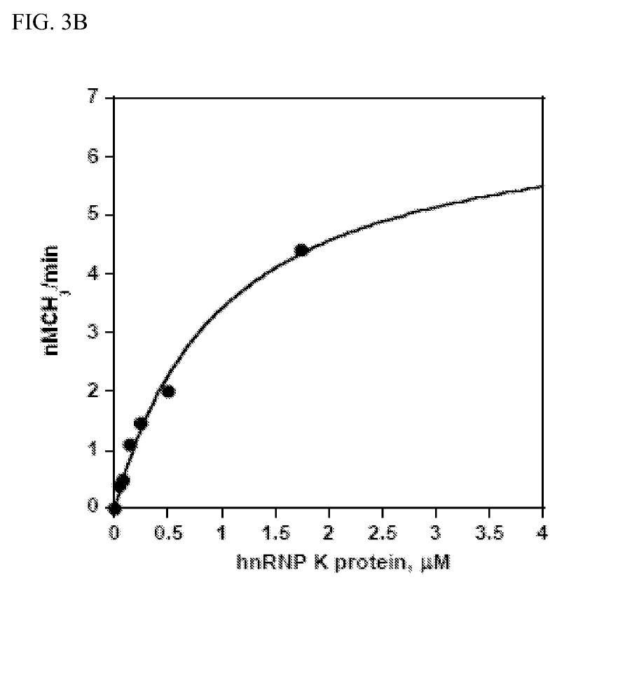

Referring now to the invention in more detail, in FIG. 1 there are shown elution profiles of radioactivity showing the separation of unreacted [³H]-AdoMet from incorporated ³H-methyl using pipette tips containing reversed-phase (C4) chromatography resin fixed at the tip. 1.7 μM hnRNP K was incubated with 1.0 μM unlabeled AdoMet, 0.6 μCi/μl [³H]-AdoMet, 100 mM Hepes, 0.38 μM BSA, 10% glycerol, 10 nM MTAN and 100 nM hPRMT1v1, at 37° C. for 12 min. A 10 μL aliquot was loaded onto pipette tips containing reversed-phase (C4) chromatography resin fixed at the tip, using a pipette. Unreacted [³H]-AdoMet was removed using 17 washes and the methylated protein eluted using organic solvent. FIG. 1A shows the disintegrations per minute (DPM) for fraction numbers one through nine. The DPM for each fraction is shown as a solid circle on the graph. FIG. 1B shows the profile from the control reaction, which substantially lacks radioactivity in fractions 5 and 6. For FIG. 1B, the DPM for each fraction is shown as an open circle on the graph. FIG. 3C is a zoomed plot of fractions 5 and 6 from FIGS. 1A and 1B, with the DPM from FIG. 1A again shown as solid circles and the DPM from FIG. 1B again shown as open circles. When the control reaction was run without protein substrate, the profile was the same.

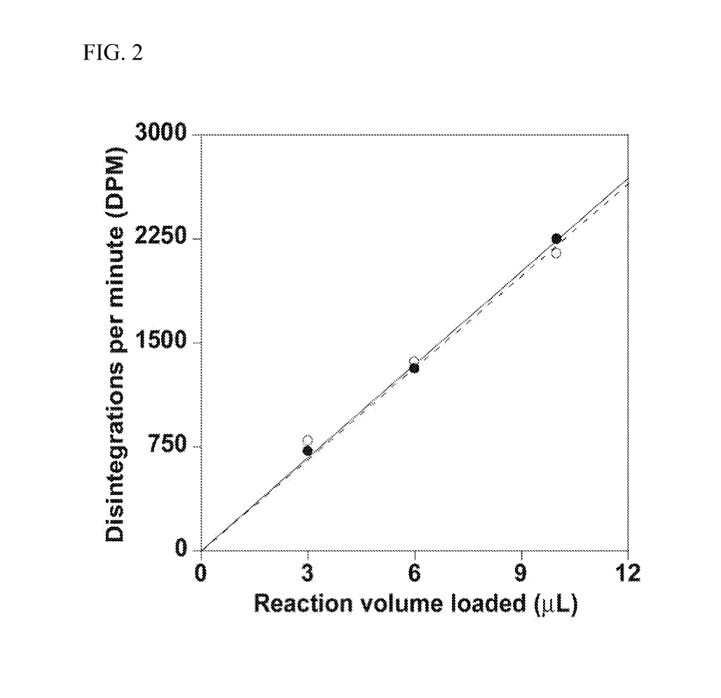

Referring now to FIG. 2, there are shown the binding and elution efficiencies of C4 reverse phase chromatography resin packed pipet tips. A sample of radiolabeled, methylated hnRNPK was prepared and incubated at 37° C. for 1.5 hours with radiolabeled AdoMet and hPRMT1v1. The sample was rapidly desalted on a DG-10 column (Bio-Rad) to remove most of the unreacted AdoMet and then further dialyzed in buffer containing 6 M Guanidinium hydrochloride and 100 mM Hepes at pH 8.0. The dialysis buffer (1 L) was changed three times. Volumes (3, 6 and 10 L) of the dialyzed protein were counted directly in scintillation cocktail (representing the total amount of methylated protein as depicted as the solid circles and a solid line). Additional volumes (3, 6 and 10 L) were applied to three pipette tips containing reversed-phase (C4) chromatography resin fixed at the tip. For example, ZipTips were used. Methylated protein eluting from the pipette tips containing reversed-phase (C4) chromatography resin fixed at the tip was counted as described above (representing the amount of methylation protein retrieved from the column and depicted as the open circles and a dotted line). Still referring to FIG. 2, all the radiolabeled protein applied to the column was retrieved from the resin packed pipet tips. Recovery of radiolabeled protein is linear over a range of protein concentrations. Optionally, 75% acetonitrile in 0.1% TFA solution is used to elute radiolabeled protein from the resin packed pipet tips. In order to determine if complete elution of Radiolabeled protein products was obtained, an additional elution step using 100% acetonitrile in 0.1% TFA was performed. No additional radioactivity was observed in this additional elution step. This result suggests that the conditions described herein are sufficient for complete binding and elution of hnRNPK protein products.

In further detail, referring now to FIG. 3, there is shown the quantitative measurement of the rate of hPRMT1v1-catalyzed methylation of hnRNP K protein using the method outlined in scheme 2. Reaction mixtures contained 1.0 μM unlabeled AdoMet, 0.6 μCi/μl [³H]-AdoMet, 100 mM Hepes, 0.38 μM BSA, 10% glycerol, and 10 nM MTAN; pH 7.6 in a final volume of 125 μl. Reactions were carried out at 37° C. Each reaction was equilibrated at 37° C. for 3 min and then initiated with 100 nM hPRMT1v1. Samples (10 μL) were removed at specified times and processed using the method shown in scheme 2. Referring now to FIG. 3A, there is shown the rate of methylation of 1.7 μM hnRNP K protein by hPRMT1v1. Referring now to FIG. 3B, there is shown a Michealis-Menten plot showing the methylation of hnRNP K protein by hPRMT1v1 as a function of hnRNPK concentration.

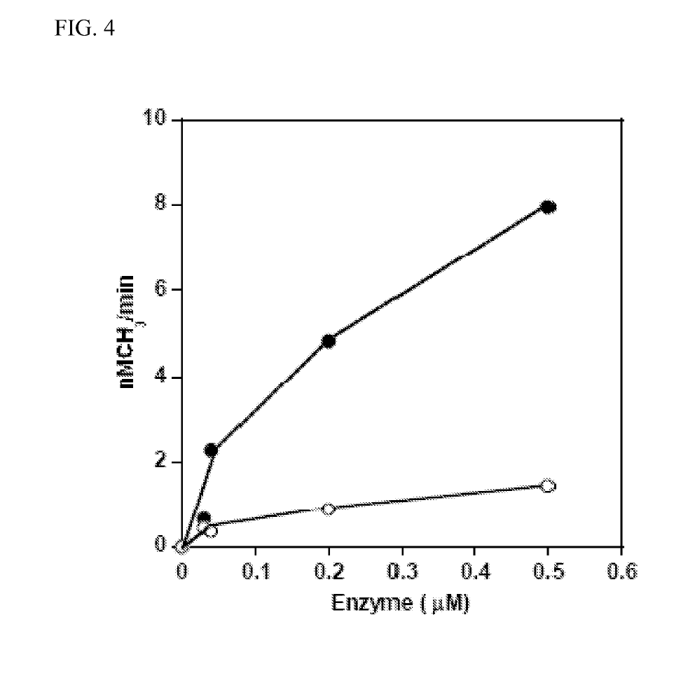

Referring now to FIG. 4, there is shown the comparison of the methylation activities of GST-PRMT6 and His-PRMT6 using the method disclosed in scheme 2. 1.7 μM Histone H3.3 was incubated with increasing amounts (0.03-0.5 μM) of GST-PRMT6 (open circles) or His-PRMT6 (closed circles), and 1.0 μM unlabeled AdoMet, 0.6 μCi/μl [³H]-AdoMet, 100 mM Hepes, 0.38 μM BSA, 10% glycerol, and 10 nM MTAN; pH 7.6 in a final volume of 125 μl. Reactions were carried out at 37° C. Each reaction was equilibrated at 37° C. for 3 min and then initiated with the appropriate amount of PRMT6. The reactions were processed and counted in scintillation cocktail.

### Examples and Embodiments

The following description and discussion of the present invention is useful in the practice of the preferred embodiments of the invention, but is not meant to imply any limitation whatsoever to the construction or practice of the present invention:

Separation of Unreacted [³H]-AdoMet from Incorporated Tritium Using Reverse Phase Chromatography Resin

Pipette tips containing reversed-phase (C4) chromatography resin fixed at the tip were used to demonstrate that free [³H]-AdoMet can be separated from the radiolabeled protein using reverse phase chromatograph resin. Methyltransferase assays were divided into two equal parts; one part was initiated with PRMT1 while the other half was initiated with water (control). Both reactions were incubated at 37° C. for 12 minutes and then were terminated. Pipette tips containing reversed-phase (C4) chromatography resin fixed at the tip were used to bind the protein in the reaction and then washed and eluted as described in the Materials and Methods section. When the bound protein was eluted, a radioactive peak was observed in the reaction initiated with hPRMT1v1 (FIG. 1A) and not with water (FIG. 1B). Baseline separation of the two peaks of radioactivity could be observed using 17 washes. This result demonstrates that pipette tips containing reversed-phase (C4) chromatography resin fixed at the tip afford a rapid and easy way to separate unreacted S-adenosyl-L-methionine from methylated protein products.

### Comparison of the Disclosed Reverse Phase Chromatography Resin Method to Currently Used Methods

We compared commonly used methods for measuring methylation of proteins to the novel methods described herein. Two identical methyltransferase assays were set up as described in the Materials and Methods section above, but different approaches were employed in the separation of unreacted [³H]-AdoMet and/or the detection of methylation. When the reaction products were separated by old methods using SDS, transferred to a membrane and the membrane incubated with film, it took 10 weeks for methylation to be detected by fluorography. A two week exposure resulted in a blank film. This shows that the sensitivity of this old method is low. Higher sensitivity can be obtained by increasing the specific activity of the [³H]-AdoMet, but one is limited by the acid that the AdoMet is supplied in, the cost of the radiolabel, and the desire to maintain saturating levels of AdoMet in the reaction.

In the second reaction, we employed the methods related to the method disclosed in scheme 2, and in less than 15 minutes obtained the rated curve shown in FIG. 3A. Rates observed with the disclosed method were comparable to the modified electrophoresis-based method (9.6 nMCH₃/min vs. 6.1 nMCH₃/min). Increased counts using the disclosed method are expected because membrane-bound radiolabeled product is subject to sample adsorbent shielding during counting. A striking difference between the assays was the amount of radiolabeled waste generated; less than 0.3 mL of radiolabeled waste was produced with the method of scheme 2 versus 1,100 mL of contaminated electrophoresis buffers for the modified electrophoresis-based method. To further support the time efficiency of this method, we were able to generate an entire Michaelis-Menten curve of the methylation of hnRNP K protein by hPRMT1v1 in ˜70 minutes (FIG. 3B).

### Comparison of His-PRMT6 and GST-PRMT6 Activities Using the Reverse Phase Chromatograph Resin Method Disclosed Herein

The ease with which the method of the scheme 2 assay is performed allows for a more thorough investigation of AdoMet-dependent protein methylation. Accordingly, the effect of the GST tag on the activity of PRMT6 was investigated. Although the GST tag is a commonly used fusion tag in PRMT studies, investigations with PRMT1, PRMT8, and PRMT9 suggest that the N-terminus is involved in regulating methylation activity. When we employed the method of scheme 2 to measure the rate of methylation of Histone H3.3 protein by His-PRMT6 and GST-PRMT6, the activity of the two fusion proteins differed greatly. It is important to note that both His-PRMT6 and GST-PRMT6 were purified in the same length of time with similar steps. Two identical assays were initiated with His-PRMT6 and GST-PRMT6. As shown in FIG. 4, His-PRMT6 has 5-fold higher activity compared to GST-PRMT6 at 500 nM enzyme. It is likely the GST tag affects the activity of PRMT6, which may be due to that fact that GST can form dimers that may affect the oligomeric structure of PRMT6.

### Additional Embodiments

In one embodiment, applicant's novel and improved method to measure methyltransferase activity can be used to quantitate AdoMet-dependent protein methylation as exemplified by the PRMT family of enzymes described in this application. One skilled in the art can easily envision embodiments of the assay for other AdoMet-dependent protein methyltransferases such as lysine methyltransferases and carboxylmethyltransferases, and may adjust certain reaction components to optimize the results of the method, according to the methyltransferase used.

Without wishing to limit the scope of the invention, one embodiment of the present invention is the measuring of protein methylation, or the activation or inhibition of protein methylation. The measuring of protein methylation, or the activation or inhibition of protein methylation, may be useful in the screening of potential therapeutic compounds. Other embodiments include measuring nucleic acid methylation or small molecule methylation.

While the foregoing written description of the invention enables one of ordinary skill to make and use what is considered presently to be the best mode thereof, those of ordinary skill will understand and appreciate the existence of variations, combinations, and equivalents of the specific embodiment, method, and examples herein. The invention should therefore not be limited by the above described embodiment, method, and examples, but by all embodiments and methods within the scope and spirit of the invention as claimed.

I claim:

1. A method of measuring methyltransferase activity, comprising the following steps:
i) incubating a mixture comprising a methyltransferase, a substrate, and a radiolabeled S-adenosylmethionine for a period of time sufficient for the methyltransferase to transform at least some of the substrate into a methylated product of the substrate,
ii) at one or more times contacting an aliquot of the mixture to a resin capable of binding the methylated product of the substrate or separating the methylated product of the substrate from other components of the mixture,
iii) optionally washing the resin to remove unreacted radiolabeled S-adenosylmethionine,
iv) optionally eluting the methylated product of the substrate from the resin,
v) measuring the amount of radiolabel in the methylated product,
vi) optionally correlating the amount of radiolabel in said methylated product to the amount of the methylated product.

2. The method of claim 1, wherein said resin is selected from a group comprising: reverse phase resin, ion exchange resin, and gel filtration resin.

3. The method of claim 1, wherein said measuring the amount of radiolabel in the methylated product is by scintillation counting on a LSC.

4. The method of claim 1, wherein said measuring the amount of radiolabel in the methylated product is by spotting the methylated product of on a membrane and developing the membrane by autoradiography.

5. The method of claim 1, wherein said measuring the amount of radiolabel associated with the methylated product is by proximity assay.

6. The method of claim 1, wherein said resin is contained in a pipette tip or multi-well plate.

7. The method of claim 1, wherein said methyltransferase is a protein methyltransferase.

8. The method of claim 7, wherein said methyltransferase is capable of methylating an amino acid from a group comprising lysine, histidine, asparagines, glutamine, and arginine.

9. The method of claim 1, wherein said incubating a mixture comprising a protein methyltransferase, a substrate, and radiolabeled S-adenosylmethionine is done for a period of time between 0 and 12 minutes.

10. The method of claim 1, wherein said contacting an aliquot of the mixture to a resin capable of binding to methylated substrate is done for a period of time less than 2 minutes.

11. The method of claim 1, wherein said substrate is a member of a group comprising small molecules, peptides, proteins, and nucleic acids.

12. The method of claim 1, wherein said substrate is a protein.

13. The method of claim 1, wherein said substrate is a peptide.

14. The method of claim 1, wherein said methyltransferase is a PRMT and said S-adenosylmethionine is S-adenosyl-L-methionine.
